(19) United States
(12) Patent Application Publication
(10) Pub. No.: US 2023/0404821 A1
(43) Pub. Date: Dec. 21, 2023

(54) NOVEL COMPOSITIONS AND USES OF MODIFIED SUPERABSORBENT POLYMER (SAP) AND OTHER GRANULAR SUBSTRATES RESULTING FROM THE INTEGRATION OF ANTIMICROBIAL AND MALODOR-CONTROLLING PROPERTIES

(71) Applicant: MedeSol Global, Inc., Bellevue, WA (US)

(72) Inventors: Jeffrey F. Williams, Langley, WA (US); Shelby D. Worley, Auburn, AL (US)

(21) Appl. No.: 18/316,512

(22) Filed: May 12, 2023

Related U.S. Application Data
(60) Provisional application No. 63/342,299, filed on May 16, 2022.

(51) Int. Cl. A61F 13/53; A61F 13/49; A61F 13/84; A61L 15/60; A61L 15/24; A61L 15/46; A61L 15/18

(52) U.S. Cl. CPC A61F 13/53; A61F 13/49; A61F 13/8405; A61L 15/60; A61L 15/24; A61L 15/46; A61L 15/18; A61F 2013/49076; A61F 2013/530481; A61L 2101/44

(57) ABSTRACT

This invention discloses novel particulate compositions made by modifying Superabsorbent Polymer (SAP) and other high surface area substrates to create granules that exhibit broad-spectrum antimicrobial and enzyme-inactivating properties, and that can directly neutralize certain malodorants and other undesirable chemical entities, such as toxins. This is accomplished by the direct interaction of the modifying moieties with: (1) vital components of bacteria, fungi, yeasts, and viruses; (2) enzymes, such as those that are responsible for the generation of malodorants and other toxic volatile compounds; and (3) certain malodorants and other undesirable compounds that are susceptible to oxidative or halogenation reactions that can neutralize their odors or toxicities. Chemical modifications of granular materials are accomplished by exposure to organic solvents containing soluble or dispersible N-halamine.

This application claims benefit of U.S. Provisional Patent Application Ser. No. 63/342,299, filed on May 16, 2022. The teachings of U.S. Provisional Patent Application Ser. No. 63/342,299 are incorporated herein by reference in their entirety.

### TECHNICAL FIELD

The present disclosure relates generally to the methods of creating particles that can be used for the control of malodors by their direct chemical neutralization, by the inactivation of contaminating microbes responsible for production of malodorants, and by the inactivation of microbial exoenzymes that produce volatile malodorants from chemical substrates in biological fluids. Odor control may come about as a result of introduction of these properties into Superabsorbent Polymers that are used in a wide variety of consumer and medical devices to absorb urine and other bodily secretions and excretions, including wound exudates. It may also come about from the introduction of modified high surface-area granules of many types into filtration or absorptive products intended for the purification of air and gas, into absorptive media that find use in animal litter, or in biological fluid or toxic spill neutralization, and as absorbents in perishable food packaging.

### BACKGROUND OF THE INVENTION

More than two million metric tonnes of Superabsorbent polymers (SAP) are produced annually (Meshram et al, 2020), the great majority (>95%) in granular form for incorporation into consumer devices such as incontinence diapers, mattress and furniture pads, feminine hygiene products, and infant diapers. The absorptive capacity of these cross-linked polymer granules may allow for the uptake of as much as 100 times their weight of water, with the amount dependent on polymer structure specifications, and the nature of the biological fluid subject to absorption. Although polymerization controls in manufacturing may allow for functional differences in absorptive capacities at surface and interior components of SAP granules (Meshram et al., 2020) other modifications to introduce useful functions cannot readily be effected in industrial manufacturing processes. This constraint is due to the superabsorptive properties intrinsic to the polymers upon contact with aqueous media. No chemical alterations using non-aqueous solvent environments have been described to date, despite the obvious advantages that would come about from the incorporation of odor-controlling functionalities into media used in incontinence or personal hygiene devices; odor emanations are especially problematic both in normal use patterns and in waste stream management of soiled devices.

With a primary view to enabling the introduction of odor control chemistry into SAP granules the present invention was developed to allow for their modification at industrial scale for incorporation into absorptive devices. The agents selected for this purpose are known to be compatible with conventional organic solvents that do not result in absorption and swelling of SAP. However, they do enable the integration of reactive moieties that show powerful, broad-spectrum antimicrobial efficacy, and also result in chemical modifications of malodorants themselves, as well as microbial exoenzymes that lead to formation of volatile reaction products from substrates in biological fluids. The formulations can also be used to alter the surfaces and compositions of other high surface area granular materials to provide for odor control and other additional utilities.

U.S. Pat. No. 3,612,055 discloses a disposable diaper having an absorbent core and was assigned to Johnson & Johnson. This disposable multilayer diaper has a high degree of absorptive capacity and is comprised of a first layer, to be brought into contact with an infant's skin, a porous, fibrous, nonwoven bonded facing web of controlled wettability made of mixed long and short fibers. A second layer, in juxtaposition to the facing layer, is a highly porous, loosely compacted cellulosic batt having greater wettability than that of the facing web. A third layer, integral with the second, is a paperlike, densified, highly compacted layer of the same cellulosic material as the second layer but of substantially smaller average pore size. The final layer is an impervious backing sheet adhered to the densified layer over a widely distributed area of adhesion. U.S. Pat. No. 3,612,055 more specifically reveals a multilayer diaper comprising (1) a porous facing layer in the form of a water-wettable bonded web of mixed long and short fibers, (2) a highly porous, loosely compacted, cellulosic fibrous batt in face-to-face juxtaposition to said facing layer and having greater wettability to water than said facing layer, (3) a paperlike, densified compacted cellulosic fibrous layer of relatively high wettability and relatively high fluid retentivity integral with said loosely compacted batt on the face thereof opposite the face in juxtaposition to said facing layer, and (4) a water-impervious backing sheet adhered to said densified layer.

U.S. Pat. No. 4,226,238 discloses a diaper comprises an outer sheet of waterproof plastic material having a deformed recess area between the sides and ends thereof which forms a pad receiving cavity which is bordered by marginal areas on each side and end. The cavity is about an inch deep and includes a bottom which is advantageously provided with corrugations or pleats which facilitate its expansion or distortion during wearing. An absorbent pad is disposed in the cavity, and a moisture holding strip of plastic material overlies at least a part of the marginal area on each side of the cavity and at least a portion of each side of the pad. The strip is welded to the marginal areas, and as a feature of the construction the plastic is stretched relative to the outer sheet and held in a stretched condition during welding so as to impart some resilient characteristics to the area which is adapted to be engaged around the legs of the wearer. Because the strip overlies the cavity, it aids in securing the pad in position and it ensures that any liquid such as urine which may collect in the pad will not run out of the cavity.

U.S. Pat. No. 4,402,690 describes a diaper which includes include a central layer of highly absorbent material. This patent more specifically reveals a diaper for children comprising: a main body having inwardly and outwardly facing surfaces, a first longitudinal end, a second longitudinal end, a first side edge and a second side edge; a first pair of oppositely directed laterally extending wings formed on said first end; a second pair of oppositely directed laterally extending wings formed on said second end and adapted to overlap said first pair of wings; hook and pile fastener means connected to said first and second pairs of wings and comprising a hook section and a pile section for maintaining said wings in overlapped disposition to form said diaper to the torso of an infant; wherein said hook section is disposed on said outwardly facing surface of said first pair of wings and said pile section is disposed on said inwardly facing surface of said second pair of wings; and elastic stretch means attached longitudinally of said body for gathering portions of said body about the legs of an infant; wherein said main body and said first and second pairs of wings are formed in a unitary construction from a plurality of layers comprising plies of dissimilar materials; wherein said layers include an inner layer of soft, nonabsorbent material for contacting an infant's skin; wherein said layers include a central layer of highly absorbent material; and further including a thick nonabsorbent inner padding layer disposed between said central layer and said inner layer for channeling moisture from said inner layer to said central layer; and an outer layer of nonabsorbing material and a thick nonabsorbing outer padding layer disposed between said central layer and said outer layer for insulating said outer layer from moisture contained in said central layer and further including quilt stitches running longitudinally of said main body for channeling moisture along said body.

U.S. Pat. No. 4,790,836 a reveals a disposable diaper for application to an infant in contact with the infant's skin, comprising: a liquid absorbent core; a continuous, homogeneous water-soluble film which dissolves upon contact with water, said film being carried by said liquid absorbent core on one side thereof to be applied in contact with the infant's skin; and a continuous, homogeneous layer of medicated powder applied as a separate and distinct layer between said water soluble film and said liquid absorbent core.

U.S. Pat. No. 4,883,479 which has been assigned to Colgate-Palmolive Company describes an absorbent article comprising a base sheet of fluid impervious material, a top sheet of fluid pervious non-woven material, said sheets being connected to each other about the periphery, and an absorbent pad disposed between said top sheet and said base sheet, said absorbent pad having a central portion and two side panels, said side panels being folded under said top sheet and over said central panel, and a binder on said central panel securing said side panels to said central panel to render said absorbent pad more absorbent, said absorbent pad including a wadding sheet, said binder penetrating said wadding sheet binding said side panels to said central panel.

U.S. Pat. No. 6,521,087 discloses a method for forming a diaper comprising a web of superabsorbent organic particles and individual absorbent cellulosic fibers comprising: (a) providing binder-coated particles comprising (i) about 3-80 percent by weight of the superabsorbent organic particles based on the weight of the superabsorbent organic particles and the individual absorbent cellulosic fibers; and (ii) from 0.01 to 5% by weight based on the weight of the superabsorbent organic particles, of a non-polymeric organic binder having a volatility less than water, wherein said binder comprises a glycol and is in an inactive state; said binder-coated particles being essentially free of fibers; (b) providing about 20-97 parts by weight of the individual absorbent cellulosic fibers; (c) activating the binder on said binder-coated particles from said inactive state, forming activated binder-coated particles, by doing at least one of the following: (i) applying heat to the binder coated particles, (ii) providing water in the form of a liquid, steam or a moisture laden gas to the binder coated particles; (iii) applying kinetic energy to the binder coated particles: (iv) applying kinetic energy to the individual absorbent˜mt cellulosic fibers; in the presence of the binder coated particles, or (v) adding the binder coated particles to the individual absorbent cellulosic fibers while water in the form of a liquid, steam or a moisture laden gas is in contact with the individual absorbent cellulosic fibers; (d) contacting said activated, binder-coated particles with said individual absorbent cellulosic fibers, under conditions effective to bind at least a portion of said binder-coated particles, in discrete particulate form, to said individual absorbent cellulosic fibers; (e) forming an airlaid web of said individual absorbent cellulosic fibers and said binder-coated particles; (f) compressing said web to form a densified absorbent web; and (g) assembling said densified absorbent web, cover sheet, and a backing sheet to form a diaper.

U.S. Pat. No. 6,630,611 describes absorbent elements for hygiene articles. This patent more specifically describes an absorbent element for a disposable absorbent hygiene article for absorbing and storing body fluids, notably urine. The element includes an absorbent and distributing layer which, during use, is situated in proximity to the body and contains internally cross-linked cellulose fibers, and a storage layer which, during use, is situated away from the body and contains natural, non-cross-linked cellulose fibers and superabsorbant materials. The horizontal expansion of the absorbent and distributing layer situated close to the body is less than that of the storage layer and in all directions does not extend beyond the edges of the storage layer. The absorbent and distributing layer consists of internally cross-linked cellulose fibers having a first retention value of between 0.6 and 0.9 g_{η}/_{Fiber} and between 8 and 15% by weight superabsorbant polymer materials and the storage layer consists of non-cross-linked cellulose fibers having a second retention value of between 1.0 and 1.4 g_{η}/fiber and of at least 20% by weight superabsorbant polymer materials.

### SUMMARY OF THE INVENTION

The integration of compounds that contain oxidative forms of chlorine (Cl) into and onto inanimate surfaces, whether soft or hard, is known to confer onto the modified substrates properties associated with oxidation and halogenation of susceptible chemical structures (see U.S. Pat. No. 10,178,866 to Worley et al.) This can lead to powerful antimicrobial activity, as well as the degradation of malodorous compounds and the inactivation of enzymes, toxins and other undesirable compounds that come into contact with the modified surfaces. According to this invention the selective use of halogen-containing compounds that are soluble or readily dispersible into organic solvents, such as ethanol, methanol, isopropanol and others, uniquely allows for the exposure and modification of Superabsorbent polymer (SAP) granules so as to confer highly useful properties upon them. Conferral of these properties is not achievable by conventional water-based methods due to the superabsorbent and rapid swelling characteristics of SAP in water. In addition to avoidance of these changes, the use of organic solvents for modification of SAP allows for rapid removal of solvent carriers by evaporation, with their possible recovery and re-use. This advantage extends to the functional modification with halogen-containing compounds of other granular substrates that do not show superabsorbency, but for which removal of the aqueous vehicle after exposure to modifying solutions or dispersions would nevertheless prove costly and impractical.

Organic solvent-compatible compounds that can be used for these purposes include 1-chloro-2,2,5,5-tetramethyl-4-imidazolidinone or other structures that exhibit the requisite compatibility with organic solvents. Modification is brought about by simple exposure of the granular substrate to a spray of or immersion in organic solvent solutions or dispersions of the halogen-containing compounds for an appropriate time followed by evaporative removal of the solvent. By this means there is integration of the halogen-containing compounds into and onto the particulate substrate sufficient to provide for the expression of useful functions when the modified granules are used for the purposes of odor control, infection control, or neutralization of undesirable or toxic entities.

The subject invention specifically reveals a composition comprising: (1) a water absorbent particulate material; and (2) an N halamine compound that is compatible with organic solvents.

The present invention also discloses a method of making an antimicrobial and malodor-controlling water absorbent particulate material which comprises (1) dispersing an N halamine compound compatible with organic solvents in an organic solvent onto a water absorbent particulate material; (2) removing the organic solvent from the water absorbent particulate material.

The subject invention further reveals an article of manufacture comprising: (1) a water absorbent particulate material; and (2) an N halamine compound that is compatible with organic solvents, wherein said article of manufacture is selected from the group consisting of diapers, incontinence pads, hygiene pads, mattress covers, puppy pee pads, furniture covers, cat litter, toxic spill absorbents, and wound dressings.

The present invention also discloses an absorbent article comprising a base sheet of fluid impervious material, a top sheet of fluid pervious non-woven material, said sheets being connected to each other about the periphery, and an absorbent pad disposed between said top sheet and said base sheet, said absorbent pad being comprised of (1) a water absorbent particulate material; and (2) an N halamine compound that is compatible with organic solvents, said absorbent pad having a central portion and two side panels, said side panels being folded under said top sheet and over said central panel, and a binder on said central panel securing said side panels to said central panel to render said absorbent pad more absorbent, said absorbent pad including a wadding sheet, said binder penetrating said wadding sheet binding said side panels to said central panel.

The subject invention further discloses a method for forming a diaper comprising a web of superabsorbent organic particles and individual absorbent cellulosic fibers comprising: (a) providing binder-coated particles comprising (i) about 3-80 percent by weight of the superabsorbent organic particles based on the weight of the superabsorbent organic particles and the individual absorbent cellulosic fibers, wherein the superabsorbent organic particles are comprised of (1) a water absorbent particulate material; and (2) an N halamine compound that is compatible with organic solvents; and (ii) from 0.01 to 5% by weight based on the weight of the superabsorbent organic particles, of a non-polymeric organic binder having a volatility less than water, wherein said binder comprises a glycol and is in an inactive state; said binder-coated particles being essentially free of fibers; (b) providing about 20-97 parts by weight of the individual absorbent cellulosic fibers; (c) activating the binder on said binder-coated particles from said inactive state, forming activated binder-coated particles, by doing at least one of the following: (i) applying heat to the binder coated particles, (ii) providing water in the form of a liquid, steam or a moisture laden gas to the binder coated particles; (iii) applying kinetic energy to the binder coated particles: (iv) applying kinetic energy to the individual absorbent cellulosic fibers; in the presence of the binder coated particles, or (v) adding the binder coated particles to the individual absorbent cellulosic fibers while water in the form of a liquid, steam or a moisture laden gas is in contact with the individual absorbent cellulosic fibers; (d) contacting said activated, binder-coated particles with said individual absorbent cellulosic fibers, under conditions effective to bind at least a portion of said binder-coated particles, in discrete particulate form, to said individual absorbent cellulosic fibers; (e) forming an airlaid web of said individual absorbent cellulosic fibers and said binder-coated particles; (f) compressing said web to form a densified absorbent web; and (g) assembling said densified absorbent web, cover sheet, and a backing sheet to form a diaper.

### BRIEF DESCRIPTION OF THE DRAWINGS

FIG. 1 demonstrates the inhibitory effects of granules treated with 1-chloro-2,2,5,5-tetramethyl-4-imidazolidinone (MC) on urease activity.

FIGS. 2A and 2B demonstrate the effects of exposure to MC-treated particles on 3M3 MB over time.

FIG. 3 illustrates absorptive capacity before and after MC treatment.

### DETAILED DESCRIPTION OF THE INVENTION

In order to modify certain particulate substrates so as to enable their usefulness in consumer and medical product applications for odor control, as well as for other chemical reactions with microbes, microbial enzymes and toxic or undesirable compounds, the integration of components containing oxidative halogens is brought about by this invention. Exposure of the substrates to a solution or dispersion of the halogen-containing compounds by spraying or by direct immersion in the solution or dispersion results in the deposition of active halogen on and within the particulates. Upon drying the presence of the oxidative moieties on the granular materials provides for stable expression of new functions such as odor control, and other reactivities associated with oxidation and/or halogenation of target molecules that are practically useful. For example, modification of SAP particles becomes possible without triggering absorption and swelling, such that the incorporation of these into devices that are used for the absorption of bodily fluids, secretions, excretions and exudates (e.g., incontinence diapers and pads, hygiene pads, diapers, wound dressings) leads to effective control of malodors.

Modified particulates that are not capable of superabsorption but nevertheless commonly find application for their ready uptake of fluids such as urine in cat litter, or blood and potentially infectious bodily fluids taken up by spill media, can be counted upon both for odor control and for the inactivation of pathogens, or other toxic elements in spilled fluids. Bringing about useful modifications of granular forms of zeolite, bentonite, vermiculite, natural and synthetic clays, organic absorbents of vegetable origin, such as corn cob granules, and the like by simple exposure to the active constituents in organic solvents followed by drying by evaporation makes these processes practical at scale by avoiding the costs and inconveniences of driving off water when aqueous solutions or dispersions are relied upon.

Compounds suitable for bringing about these modifications include certain N-halamines that are stable in the presence of organic solvents, in particular 1-chloro-2,2,5,5-tetramethyl-4-imidazolidinone (MC). Shelf stable solutions of such compounds can be made at concentrations that are practical in conferring new properties on particulates under conditions of normal room temperature and pressure followed by evaporative removal of the solvents. Dried modified particulates can be manufactured at scale and stored for eventual use as components of devices such as incontinence diapers, mattress covers, puppy pads, cat litter, wound dressings, and spill control media.

1-chloro-2,2,5,5-tetramethyl-4-imidazolidinone (MC) can be manufactured to a high degree of purity at industrial scale by the process described in T. Tsao, D. E. Williams, C. G. Worley, and S. D. Worley, “Novel N-Halamine Disinfectant Compositions”, Biotech. Progr., 7, 60 (1991). The teachings of T. Tsao, D. E. Williams, C. G. Worley, and S. D. Worley, “Novel N-Halamine Disinfectant Compositions”, Biotech. Progr., 7, 60 (1991) are incorporated herein by reference. MC has the following structure:

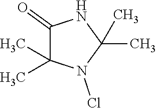

MC is powerfully antimicrobial and its use in organic solvents has been shown to confer this property upon fibrous media to create novel fibrous compositions that are useful as components of air filters, facemasks, wound dressings, odor-controlling textiles for garments, and disinfecting wipes as described in U.S. Pat. No. 10,178,866. Fibrous compositions prepared in this fashion maintain their capacity to inactivate microbes and malodorous compounds for many months on dry storage. The teachings of U.S. Pat. No. 10,178,866 are incorporated by reference herein.

Modified SAP granules have direct inactivating efficacy against Staph aureus and E. coli in time frames (10 minutes-one hour) that are consistent with the control of odor in absorptive devices into which modified SAP has been introduced in the normal amounts and manufacturing process. Introduction of these modifications into inorganic and organic granular materials used in cat litter results in direct degradative reactions with the most malodorous of the compounds excreted in urine, and with enzymes such as ureases that generate ammonia odor from urinary urea.

Superabsorbent polymers that can be utilized in the practice of this invention are described by Isha Meshram, Vrushali Kanade, Nidhisha Nandanwar, and Pradnya Ingle, “Super-Absorbent Polymer: A Review on the Characteristics and Application, International Journal of Advanced Research in Chemical Science, Volume 7, Issue 5, 2020, PP 8-21. The teachings of by Isha Meshram, Vrushali Kanade, Nidhisha Nandanwar, and Pradnya Ingle, “Super-Absorbent Polymer: A Review on the Characteristics and Application, International Journal of Advanced Research in Chemical Science, Volume 7, Issue 5, 2020, PP 8-21, are incorporated by reference herein for the purpose of describing such superabsorbent polymers. In any case, this paper explains that superabsorbent polymers (SAP) are a class of polymers that are able to absorb much more of water than typical absorbent materials. Such SAPs normally consist of a network of polymer chains which are cross-linked to avoid dissolution and they are normally functionalized with an ionic functional group along their polymer chain to facilitate diffusion of water within the network. An acrylate monomer is the principal monomeric material in synthesizing SAPs. For instance, superabsorbent poly acrylates are frequently prepared by polymerizing acrylic acid with a crosslinking agent. When water comes into contact with one of these chains, it is drawn into the molecule by osmosis. Water rapidly migrates into the interior of the polymer network where it is retained. The type and degree of cross linkers govern the ability of the superabsorbent polymer to absorb and retain large quantity of water. The water uptake potential of SAPs can be as high as 100,000% of its own weight and the water can be absorbed in a short period of time.

The monomers useful for making superabsorbent polymer are water soluble monomers, such as acrylic acid (AA), methacrylic acid, and 2-acrylamide (DAA) 2-methylpropanesulfuric acid are the principal ionizable monomers useful for making superabsorbent polymers. The useful cross-linkers include a variety of multifunctional monomers. They can be di-, tri-, or tetra functional, and can have mixed type of polymerizable groups, such as methacrylate.

U.S. Pat. No. 8,466,288 describes a superabsorbent polymer with high permeability. This SAP is a crosslinked polymer which is based on partly neutralized, monoethylenically unsaturated monomers carrying acid groups, and have improved properties, in particular in respect of their capacity for transportation of liquids in the swollen state, and which has a high gel bed permeability and high centrifuge retention capacity. This patent more specifically describes particulate superabsorbent polymer composition comprising: (a) from about 55 to about 99.9 weight percent of a monomer, which contain carboxyl groups including acrylic acid, methacrylic acid, or mixtures of these monomers wherein the monomer is neutralized to from 50 to 80 mole percent; (b) from about 0.001 to about 3.0 weight percent based on the weight of (a) of internal crosslinking agent; wherein elements (a) and (b) are polymerized into a polymer gel which is then dried in a forced air oven to have a moisture content of less than weight percent, and comminuted into particulate superabsorbent polymer composition having a particle size of from 150 to 850 μm and a particle surface and further comprising (c) from about 0.001 to about 5.0 weight percent based on a dry polymer powder weight of an alkylene carbonate applied to the particle surface in a surface crosslinking step; (d) from about 0.01% to about 5 weight percent based on the dry polymer powder weight of polyethylene glycol or tetraethyleneglycol dimethylether added immediately prior to or during the surface crosslinking step; (e) from about 0.01 weight percent to about 5 weight percent based on the dry polymer powder weight of a multivalent metal salt selected from aluminum sulfate on the particle surface; (f) from about 0.01 weight percent to about 5 weight percent based on the dry polymer powder weight of a water-insoluble, inorganic powder selected from kaolin, silicon dioxide, fumed silica, silicic acid, silicates, titanium dioxide, aluminum oxide, fumed alumina, calcium phosphate, or clays; and (g) from about 0.1 weight percent to about weight percent based on the dry polymer powder weight of a surfactant selected from disodium cocoamphopropionate or cocomonoethanol amide with 4.5 moles ethoxylation; wherein the composition has a degree of neutralization of more than about 25%; and a Centrifuge Retention Capacity of from about 23 g/g to about 40 g/g as determined by the Centrifuge Retention Capacity Test set forth herein and a gel bed permeability of from about 400×10⁻⁹ cm² to about 2500×10⁻⁹ cm² as determined by the Gel Bed Permeability Test set forth herein. The teachings of U.S. Pat. No. 8,466,288 are incorporated by reference herein.

U.S. Pat. Nos. 5,130,389, 5,286,827, and 10,646,612 describe SAPs that can be used in the practice of this invention and the synthesis thereof. The teaching of U.S. Pat. Nos. 5,130,389, 5,286,827, and 10,646,612 are incorporated herein by reference.

The compositions of this invention can be incorporated into a wide variety of absorbent articles including diapers, incontinence pads, hygiene pads, mattress covers, puppy pee pads, furniture covers, cat litter, toxic spill absorbents, wound dressings, and the like. For example it is extremely useful when used in manufacturing disposable diapers. For example, it can be used beneficially as the a highly water-absorbent fibrous pad or batt in diapers of the type described in U.S. Pat. No. 3,612,055. The teachings of U.S. Pat. No. 3,612,055 are incorporated herein for the purpose of describing such diapers.

U.S. Pat. No. 4,226,238 describes a diaper comprising an outer sheet of waterproof plastic material having a recessed area between the sides and ends thereof forming a pad receiving cavity bordered by marginal areas on each side and end, said cavity having a bottom spaced outwardly from said marginal areas, an absorbent pad disposed in said cavity, and a moisture holding strip of plastic overlying at least a part of a marginal area on each side of said cavity and at least a portion of each side of said pad and having at least a portion secured to the outer sheet along the length of said moisture holding strip. The composition of this invention can beneficially be used in the absorbent pad of diapers of this type. The teachings of U.S. Pat. No. 4,226,238 are incorporated herein for the purpose of describing such diapers.

U.S. Pat. No. 4,402,690 discloses a diaper for children comprising: a main body having inwardly and outwardly facing surfaces, a first longitudinal end, a second longitudinal end, a first side edge and a second side edge; a first pair of oppositely directed laterally extending wings formed on said first end; a second pair of oppositely directed laterally extending wings formed on said second end and adapted to overlap said first pair of wings; hook and pile fastener means connected to said first and second pairs of wings and comprising a hook section and a pile section for maintaining said wings in overlapped disposition to form said diaper to the torso of an infant; wherein said hook section is disposed on said outwardly facing surface of said first pair of wings and said pile section is disposed on said inwardly facing surface of said second pair of wings; and elastic stretch means attached longitudinally of said body for gathering portions of said body about the legs of an infant; wherein said main body and said first and second pairs of wings are formed in a unitary construction from a plurality of layers comprising plies of dissimilar materials; wherein said layers include an inner layer of soft, nonabsorbent material for contacting an infant's skin; wherein said layers include a central layer of highly absorbent material; and further including a thick nonabsorbent inner padding layer disposed between said central layer and said inner layer for channeling moisture from said inner layer to said central layer; and an outer layer of nonabsorbing material and a thick nonabsorbing outer padding layer disposed between said central layer and said outer layer for insulating said outer layer from moisture contained in said central layer and further including quilt stitches running longitudinally of said main body for channeling moisture along said body. It is extremely beneficial to use the composition of this invention in the layer of highly absorbent material described in U.S. Pat. No. 4,402,690. The teachings of U.S. Pat. No. 4,402,690 are incorporated by reference herein for the purpose of describing such diapers.

The compositions of this invention can also be beneficially used as the absorbent pad in the diapers described in U.S. Pat. No. 4,883,479. The teachings of U.S. Pat. No. 4,883,479 are incorporated by reference herein for the purpose of describing such diapers.

The compositions of this invention can also be beneficially used in the web of superabsorbent organic particles employed in the diapers described in U.S. Pat. No. 6,521,087. The teachings of U.S. Pat. No. 6,521,087 are incorporated by reference herein for the purpose of describing such diapers. The compositions of this invention can also be beneficially used in superabsorbent polymer materials employed in the hygiene articles described in in U.S. Pat. No. 6,630,611. The teachings of U.S. Pat. No. 6,630,611 are incorporated by reference herein for the purpose of describing such hygiene articles.

This invention is illustrated by the following examples that are merely for the purpose of illustration and are not to be regarded as limiting the scope of the invention or the manner in which it can be practiced. Unless specifically indicated otherwise, parts and percentages are given by weight.

### Example 1

### Detection of Active Chlorine in Modified Granules

This experiment was conducted to demonstrate that exposure of a variety of particulate substrates to 1% 1-chloro-2,2,5,5-tetramethyl-4-imidazolidinone (MC) in ethanol followed by air drying results in the presence of appreciable active Cl on the particulates demonstrable by visible reaction with 10% Potassium Iodide solution. 5 gm samples of SAP granules extracted from infant diapers, and granules of clinoptilolite zeolite, bentonite, vermiculite, and corn cob cellulose/lignin were exposed to the MC preparation by soaking for 10 minutes at 20° C. After air drying the samples were stored in the dark at 20° C. for 60 days. At that time drops of KI solution were added to the dried granules which in all cases showed an immediate yellow-brown deposit indicating the presence of iodine resulting from the oxidation of iodide by the chlorine in MC deposited in and on the granules.

### Example 2

### Demonstration of Titratable Chlorine in MC Modified Granules

This experiment was conducted in order to quantify the amounts of active Cl deposited in and on various granules by exposure to 1% MC solutions in ethanol and persistence of the titratable chlorine over time. In the procedure used one gram samples of SAP granules extracted from infant diaper matrix, clinoptilolite zeolite, bentonite, corn cob and GAC granules were all soaked in 1% MC in ethanol for 10 minutes at room temperature (20° C.) and air dried. Control samples were exposed to ethanol alone and air dried. After storage of the dried samples in the dark at 20° C. for up to 5 weeks the samples were extracted with water for 10 minutes with stirring and the supernatants were titrated for Cl content using the HACH digital titration reagents and procedure. Control granules of each type unexposed to MC consistently showed no reactivity with the HACH reagents. After 20-40 days samples from modified SAP granules contained 120-275 ppm of titratable Cl/mL; zeolite and bentonite contained 110 ppm and 124 ppm respectively while corn cob granules contained 86 ppm. GAC granules contained 150 ppm of titratable Cl at approximately 20 days post treatment. These results indicate that active chlorine is integrated into porous granules of various types, inorganic and organic, and persists at measurable levels for weeks while exposed to air at room temperature.

### Example 3

### Demonstration of Antimicrobial Efficacy of MC-Modified SAP

This experiment was conducted in order to show that SAP granules exposed to MC and air-dried have efficacy in a test for the inactivation of Staphylococcus aureus bacteria suspended in water. In the procedure used 0.05 gm of SAP granules that had been MC treated for 10 minutes were air dried one week previous to the experiment and kept at 20° C. Control untreated SAP and MC-treated granules were both allowed to swell in phosphate buffered saline for 30 minutes. Each preparation was then added to 1 mL of a suspension of S. aureus containing approximately one million bacteria. Both preparations were allowed to mix on a rotatory mixer at room temperature (20° C.) for 30 minutes. At the end of the incubation six 3 mm diameter sterile glass beads were added to each mixture of the SAP and bacterial suspension and vortexed vigorously for 1 minute. After addition of 9 mL of 0.05N sodium thiosulphate to each preparation to quench the active chlorine, samples of the supernatant were plated in serial ten-fold dilutions on nutrient agar Petri plates and incubated for 24 hours at 37° C. Samples from the control SAP granule preparation contained an average of 510,000 viable S. aureus. There was no growth in samples that had been mixed with the MC-modified SAP granules, indicating a log reduction value (LRV) of greater than 5, over the 30 minutes of exposure.

### Example 4

### Demonstration of the Inhibitory Effects of Granules Treated with MC on Urease Activity

This experiment was done to show the effect of modified SAP granules on the activity of an enzyme, urease, responsible for generating a malodorant, ammonia, from urea in urine. A 1 mg/mL stock solution of Canavalia ensiformis urease (1 U/mg) was prepared in 200 mM phosphate buffer (pH 7.0). MC-treated granules (10 mg) were dispersed in 2.825 mL of 200 mM of phosphate buffer pH 7.0 for 2 minutes prior to enzyme exposure. 175 μL of the urease stock solution (1 IU/mL) was then added and the mixture incubated for 30 minutes. The samples were vortexed for 30 seconds after the addition of urease and again after the reaction was complete. The mixture was centrifuged using a Beckman Coulter microfuge® 20R, at 5000 RPM for 2 minutes. Any remaining activity of the MC was quenched by the addition of sodium thiosulfate (1%). Then, 180 μL of the quenched reaction mixture was incubated with urea (15 mM) for 5 minutes. A 100 μL sample of the supernatant was tested following the Sigma Aldrich Ammonia Assay Kit protocol (AA0100).

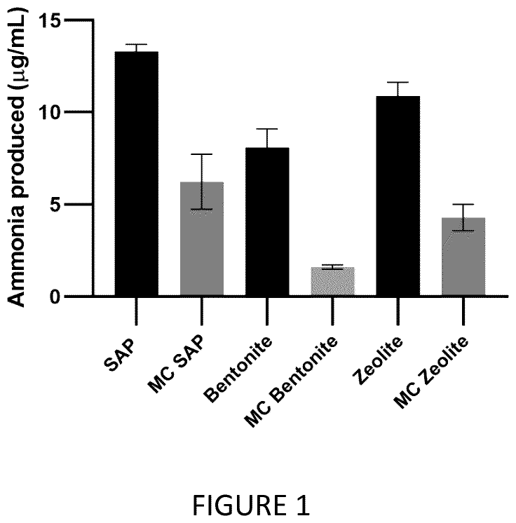

MC-treated bentonite was most effective at urease inhibition, reducing the activity by approximately 87%. MC-treated SAP granules and MC-treated zeolite decreased the enzyme activity by about 54% and about 70%, respectively (FIG. 1).

### Example 5

### Demonstration of the Effects of Exposure to MC-Treated Particles on 3M3 MB Over Time

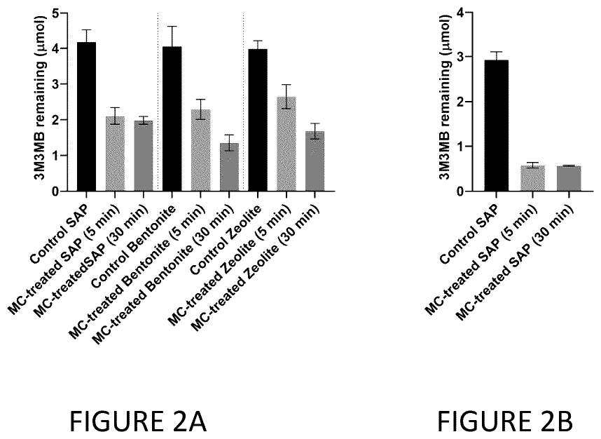

In this experiment MC-coated SAP, zeolite and bentonite were tested for their ability to modify 3M3 MB, a known odor-producing thiol in both aqueous and alcohol solutions. After 5 minutes of incubation, MC-coated SAP and bentonite reduced the amount of thiol detected by greater than 93%. In contrast, about 40% 3M3 MB was detected after 5 minutes with MC-coated zeolite. After 30 minutes, the three granule types showed similar reduction in detectable 3M3 MB (FIG. 2A). The ability of MC-coated SAP to modify 3M3 MB in ethanol (99%) where the granules are absorbing less water, showed a reduction of an about 80% after 5 minutes with no further reduction being observed after 30 minutes (FIG. 2B).

### Example 6

### Demonstration of the Absorptive Capacity Before and After MC Treatment

This experiment was conducted to show that the absorptive capacity of the modified SAP was unaffected by the MC treatment. The total absorptive capacity of MC-treated and untreated SAP granules was measured following a slight modification of a published procedure. In the procedure used 50 mg of SAP granules were placed inside a tea filter. The weight of the empty dry tea filter with the SAP granules was recorded (W₁). The tea filter containing the granules was fully submerged in a 600 mL beaker filled completely with ultrapure water for 30 minutes. The tea filter was removed and hung to remove excess water for 30 minutes. The tea bag was lightly shaken twice to remove any excess water and the weight was recorded (W₂). The total absorptive capacity of the SAP granules was calculated using the following equation:

$$Absorptive Capacity ( \% ) = \frac{{W}_{2}}{{W}_{1}} \times 100$$

where W₂ represents the weight of the water treated tea bag containing SAP granules (g) and W₁ represents the weight of dry tea bag and SAP granules (g).

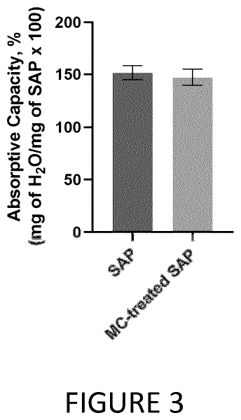

To ensure that the coating procedure did not negatively impact the SAP granules, the absorptive capacity of the MC-treated SAP granules was compared with the control SAP granules. One mg of SAP and MC-treated SAP absorbed about 155 mg and 148 mg of water, respectively. No significant difference was found after treating SAP granules with MC (FIG. 3).

### Example 7

### Demonstration of Scalability of the Modification Process and the Utility of Modified SAP in Controlling Malodor in Adult Incontinence Diapers

In this experiment 50 Kg of SAP were exposed to a 2% N halamine solution in methanol for 30 minutes, and air dried before incorporation into adult incontinence devices at the rate of 13 grams of modified SAP per unit. SAP modification was performed at Dimachem, Windsor. Ontario. Canada. and the treated granules were incorporated into the absorptive core of adult incontinence devices at Winsun, Corporation. Foshan City, Guangdong Province, China. Finished devices containing the modified SAP were transported to Seattle WA. USA for evaluation by wearers.

First two healthy adult male volunteers wore the diapers made by Winsun for 8 hours in order to ensure that prolonged contact with the devices in normal use did not cause any adverse effects on skin making contact with the inner aspect of the urine-soaked devices over time. Since there was no such adverse effect, the units were then used by six urinary incontinent female adults replacing their usual daily use of commercial diaper devices with diapers made at Winsun which contained the modified SAP granules. Over the course of a day to five days these individuals used several (up to six) of the Winsun products per day, and either reported their assessment of the effect of the devices on odor compared to their normal experience at the end of each wear cycle, or the effect of the devices on malodor was recorded by their home care-givers.

In all instances the users and/or their care-givers reported a high level of inhibition of malodor development normally present at the end of each wear cycle. In one instance where fecal incontinence accompanied urinary incontinence the care-giver reported satisfactory inhibition of fecal malodor at the end of a wear cycle. It was further noted that upon storage of the used diapers prior to their disposal there was a high level of inhibition of odor production in the storage bins.

These observations indicate that the modification process can be reliably applied to SAP granule batches at industrial scale. Furthermore, the results show that integration of modified SAP granules into the absorptive core of adult incontinence devices leads to noticeable and beneficial reduction in the amount of malodorants generated in normal routine use compared to commercially available diapers containing unmodified SAP granules.

While certain representative embodiments and details have been shown for the purpose of illustrating the subject invention, it will be apparent to those skilled in this art that various changes and modifications can be made therein without departing from the scope of the subject invention.

What is claimed is:

1. A composition comprising: (1) a water absorbent particulate material; and (2) an N halamine compound that is compatible with organic solvents.

2. The composition of claim 1 wherein the halogen containing compound is 1-chloro-2,2,5,5-tetramethyl-4-imidazolidinone.

3. The composition of claim 1 wherein the water absorbent particulate material is comprised of superabsorbent polymer particles.

4. The composition of claim 3 wherein the superabsorbent polymer particles are comprised of an acrylic polymer.

5. The composition of claim 3 wherein the superabsorbent polymer is comprised of at least one monomer selected from the group consisting of acrylic acid, methacrylic acid, and 2-acrylamide-2-methylpropanesulfuric acid.

6. The composition of claim 5 wherein the superabsorbent polymer is crosslinked.

7. The composition of claim 3 wherein the water absorbent particulate material is selected from the group consisting of zeolite, bentonite, vermiculite, natural and synthetic clays, laponite, granulated carbon, corn cob cellulose and lignin particulates, and organic absorbents of vegetable origin.

8. The composition of claim 1 wherein the composition contains from about 50 ppm to about 600 ppm of titratable Cl/mL.

9. The composition of claim 1 wherein the composition contains from about 100 ppm to about 300 ppm of titratable Cl/mL.

10. The composition of claim 1 wherein said composition is essentially free of water.

11. A method of making an antimicrobial and malodor-controlling water absorbent particulate material which comprises (1) dispersing an N halamine compound compatible with organic solvents in an organic solvent onto a water absorbent particulate material; (2) removing the organic solvent from the water absorbent particulate material.

12. The method of claim 11 wherein the organic solvent is recovered from the water absorbent particulate material.

13. The method of claim 12 wherein the organic solvent which is recovered is recycled for use in making additional antimicrobial and malodor-controlling water absorbent particulate material.

14. The method of claim 12 wherein the organic solvent is selected from the group consisting of methanol, ethanol, n-propyl alcohol, and isopropyl alcohol.

15. The method of claim 11 wherein the N halamine compound is present in the organic solvent at a concentration which is within the range of about 0.8 weight percent to about 2 weight percent.

16. An article of manufacture which includes the composition of claim 1 wherein said article of manufacture is selected from the group consisting of diapers, incontinence pads, hygiene pads, mattress covers, puppy pee pads, furniture covers, cat litter, toxic spill absorbents, and wound dressings.

17. The article of manufacture of claim 16 wherein the composition is incorporated into said article as a water absorbent layer.

18. An absorbent article comprising a base sheet of fluid impervious material, a top sheet of fluid pervious non-woven material, said sheets being connected to each other about the periphery, and an absorbent pad disposed between said top sheet and said base sheet, said absorbent pad comprising the composition of claim 1, said absorbent pad having a central portion and two side panels, said side panels being folded under said top sheet and over said central panel, and a binder on said central panel securing said side panels to said central panel to render said absorbent pad more absorbent, said absorbent pad including a wadding sheet, said binder penetrating said wadding sheet binding said side panels to said central panel.

19. The absorbent article of claim 18 wherein said article is a sanitary napkin or a disposable diaper.

20. A method for forming a diaper comprising a web of superabsorbent organic particles and individual absorbent cellulosic fibers comprising: (a) providing binder-coated particles comprising (i) about 3-80 percent by weight of the superabsorbent organic particles based on the weight of the superabsorbent organic particles and the individual absorbent cellulosic fibers, wherein the superabsorbent organic particles are comprised of the composition of claim 1; and (ii) from 0.01 to 5% by weight based on the weight of the superabsorbent organic particles, of a non-polymeric organic binder having a volatility less than water, wherein said binder comprises a glycol and is in an inactive state; said binder-coated particles being essentially free of fibers; (b) providing about 20-97 parts by weight of the individual absorbent cellulosic fibers; (c) activating the binder on said binder-coated particles from said inactive state, forming activated binder-coated particles, by doing at least one of the following: (i) applying heat to the binder coated particles, (ii) providing water in the form of a liquid, steam or a moisture laden gas to the binder coated particles; (iii) applying kinetic energy to the binder coated particles: (iv) applying kinetic energy to the individual absorbent cellulosic fibers; in the presence of the binder coated particles, or (v) adding the binder coated particles to the individual absorbent cellulosic fibers while water in the form of a liquid, steam or a moisture laden gas is in contact with the individual absorbent cellulosic fibers; (d) contacting said activated, binder-coated particles with said individual absorbent cellulosic fibers, under conditions effective to bind at least a portion of said binder-coated particles, in discrete particulate form, to said individual absorbent cellulosic fibers; (e) forming an airlaid web of said individual absorbent cellulosic fibers and said binder-coated particles; (f) compressing said web to form a densified absorbent web; and (g) assembling said densified absorbent web, cover sheet, and a backing sheet to form a diaper.
